(19) United States
(12) Patent Application Publication
(10) Pub. No.: US 2024/0336913 A1
(43) Pub. Date: Oct. 10, 2024

(54) METHOD FOR PRODUCING A POPULATION OF SYMMETRICALLY BARCODED TRANSPOSOMES

(71) Applicants: Derek BOGDANOFF, Berkeley, CA (US); Chang KIM, Berkeley, CA (US); Tomasz NOWAKOWSKI, Berkeley, CA (US); The Regents of the University of California, Oakland, CA (US); CHAN ZUCKERBERG BIOHUB, INC., San Francisco, CA (US)

(72) Inventors: Derek Bogdanoff, Berkeley, CA (US); Chang Kim, Berkeley, CA (US); Tomasz Nowakowski, Berkeley, CA (US)

(21) Appl. No.: 18/563,310

(22) PCT Filed: May 26, 2022
(86) PCT No.: PCT/US22/31135

§ 371 Date: Nov. 21, 2023

Related U.S. Application Data
(60) Provisional application No. 63/195,515, filed on Jun. 1, 2021.

(51) Int. Cl. C12N 15/10

(52) U.S. Cl. CPC C12N 15/1065

(57) ABSTRACT

Provided herein is a method for producing a population of symmetrically barcoded transposomes, i.e., transposomes that are loaded with a pair of identical double-stranded adapters. In some embodiments, this method may comprise amplifying a template that contains a randomized sequence and a transposon end sequence on a solid support by bridge polymerase chain reaction (PCR) to produce uniquely barcoded clusters of single-stranded amplification products that are tethered to the support, processing the single-stranded amplification products so that the transposon end sequence is double-stranded and at the end of the products, and adding transposase to the support under conditions by which the transposase binds to the double-stranded transposon end sequences, to produce population of symmetrically barcoded transposomes.

### CROSS-REFERENCE

This application claims the benefit of U.S. Provisional Patent Application No. 63/195,515, filed Jun. 1, 2021, which application is incorporated herein by reference in its entirety.

### STATEMENT REGARDING FEDERALLY SPONSORED RESEARCH

This invention was made with government support under Grant No. RF1 MH121268 awarded by National Institutes of Health. The government has certain rights in the invention.

### INCORPORATION BY REFERENCE OF SEQUENCE LISTING PROVIDED AS A TEXT FILE

A Sequence Listing is provided herewith as a text file, “CZBH-004WO_SEQ_LIST_ST25.txt” created on Apr. 18, 2022 and having a size of 2 KB. The contents of the text file are incorporated by reference herein in their entirety.

### INTRODUCTION

Many next generation sequencing workflows involve “tagmenting” (i.e., cleaving and tagging) a sample in a reaction that is catalyzed by a transposase (see, e.g., Caruccio Methods Mol. Biol. 2011 733: 241-55; Kaper et al, Proc. Natl. Acad. Sci. 2013 110: 5552-7; Marine et al, Appl. Environ. Microbiol. 2011 77: 8071-9). In such methods, double-stranded adapters that contain a transposon end sequence and a PCR amplification sequence are combined with transposase to produce transposome complexes that each contain two molecules of the double-stranded adapter. In the tagmentation reaction, the transposase introduces a double-stranded break at a site in a nucleic acid and adds an adapter to each of the cleaved ends. The fragments can be amplified using primers that hybridize to the PCR amplification sequences, and then sequenced.

The double-stranded adapters that are added to the fragments can contain a rationally designed or random barcode (see, e.g., Lau et al, BMC Genomics 2017 18: 745). In these methods, all the fragments receive different barcodes which, in turn, allows the fragments to be distinguished from one another. The problem with these methods, however, is that information about which fragments are next to each other in the unfragmented nucleic acid is lost after tagmentation. As such, it is often impossible to reconstruct the unfragmented sequence without relying on alignment to a reference sequence This disclosure addresses this problem, and others.

### SUMMARY

Provided herein is a method for producing a population of symmetrically barcoded transposome complexes, i.e., transposomes that are loaded with a pair of identical double-stranded adapters. In some embodiments, this method may comprise amplifying a template that contains a randomized sequence and a transposon end sequence on a solid support by bridge polymerase chain reaction (PCR) to produce uniquely barcoded clusters of single-stranded amplification products that are tethered to the support, processing the single-stranded amplification products so that the transposon end sequence is double-stranded and at the end of the products, and adding transposase to the support under conditions by which the transposase binds to the double-stranded transposon end sequences, to produce the population of symmetrically barcoded transposomes.

Nucleic acid molecules from different clusters cannot be bound by a single transposase molecule since they are too far away and, as such, in the binding step, the transposase molecule binds to pairs of nucleic acid molecules that are within same cluster. Because the barcode sequence is the same within each cluster, the transposome complexes produced by the present method are symmetrically barcoded. As will be described in greater detail below, these transposomes can be cleaved from the substrate and used in solution or left on the substrate and used in situ.

This method has the potential to generate hundreds of millions or even billions of clonally derived, symmetrical barcoded, dimerized transposase/DNA-adapter (“transposome”) complexes that can be used for the preparation of sequencing libraries. The transposome complexes produced by the method should contain symmetrical adapters (i.e., a pair of adapters that have the same barcode sequence). As such, during tagmentation each transposome complex should make a double-stranded break in the substrate and add the same barcode to the newly created ends. This, in turn, facilitates assembly of the sequences because adjacent fragments have the same barcode.

Also provided is a substrate comprising clusters of transposomes, wherein each transposome comprises: (a) two identical molecules of amplification product that each have a proximal end that is tethered to the support, a barcode sequence, and a distal end that comprises a double-stranded transposon end sequence, and (b) a transposase, wherein the transposase is bound to the transposon end sequences of the two molecules of amplification product. In these embodiments, the barcode sequence is the same for all of the transposomes within a cluster but different between clusters. The substrate can comprise at least 10⁶ (1M) of said clusters, for example,

Also provided is a population of symmetrically barcoded transposomes in which each transposome comprises: a transposase and two identical molecules of nucleic acid that each comprise a barcode sequence and a double-stranded transposon end sequence. wherein the population comprises at least 1,000 of said transposomes, each with a different barcode. The population comprises at least 1M of said transposomes, each with a different barcode.

The populations of symmetrically barcoded transposomes described above may be used in a variety of methods, some of which comprise combining a nucleic acid sample with a population of transposomes and a divalent cation to produce a reaction mix, and incubating the reaction mix to tagment the nucleic acid sample.

### BRIEF DESCRIPTION OF THE DRAWINGS

The skilled artisan will understand that the drawings, described below, are for illustration purposes only. The drawings are not intended to limit the scope of the present teachings in any way.

FIG. 1 illustrates the difference between a symmetrical transposome and an asymmetrical transposome.

FIG. 2 illustrates the difference between population of a symmetrical transposomes and population of an asymmetrical transposomes.

FIG. 3 illustrates how clusters of amplification products are produced on a support.

FIG. 4 illustrates an example of a template (SEQ ID NO: 1).

FIG. 5 illustrates some of the principles of the present method.

FIG. 6 illustrates one way to generate population of a symmetrical transposomes.

FIG. 7 illustrates how symmetrical transposomes can be made on a flow cell.

FIG. 8 illustrates how the barcodes can be used for sequence assembly.

FIG. 9 shows the structure of various starting products, intermediate reaction products, and products, that may be used or made in practicing the method.

### DEFINITIONS

Unless otherwise defined, all technical and scientific terms used herein have the same meaning as commonly understood by one of ordinary skill in the art to which this invention belongs. Still, certain elements are defined for the sake of clarity and ease of reference.

Terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, greater than 10,000 bases, greater than 100,000 bases, greater than about 1,000,000, up to about 10¹⁰ or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylenecarbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid,” or “UNA,” is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

The term “nucleic acid sample,” as used herein, denotes a sample containing nucleic acids. Nucleic acid samples used herein may be complex in that they contain multiple different molecules that contain sequences. Genomic DNA samples from a mammal (e.g., mouse or human) are types of complex samples. Complex samples may have more than about 10⁴, 10⁵, 10⁶ or 10⁷, 10⁸, 10⁹ or 10¹⁰ different nucleic acid molecules. Any sample containing nucleic acid, e.g., genomic DNA from tissue culture cells or a sample of tissue, may be employed herein.

The term “oligonucleotide” as used herein denotes a single-stranded multimer of nucleotides of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) or deoxyribonucleotide monomers, or both ribonucleotide monomers and deoxyribonucleotide monomers. An oligonucleotide may be 10 to 20, 21 to 30, 31 to 40, 41 to 50, 51 to 60, 61 to 70, 71 to 80, 80 to 100, 100 to 150 or 150 to 200 nucleotides in length, for example.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers are extended by a DNA polymerase. Primers are generally of a length compatible with their use in synthesis of primer extension products, and are usually in the range of 8 to 200 nucleotides in length, such as 10 to 100 or 15 to 80 nucleotides in length. A primer may contain a 5′ tail that does not hybridize to the template.

Primers are usually single-stranded for maximum efficiency in amplification, but may alternatively be double-stranded or partially double-stranded. Also included in this definition are toehold exchange primers, as described in Zhang et al (Nature Chemistry 2012 4: 208-214), which is incorporated by reference herein. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

The term “duplex,” or “duplexed,” as used herein, describes two complementary polynucleotide regions that are base-paired, i.e., hybridized together.

“Genetic locus,” “locus,”, “locus of interest”, “region” or “segment” in reference to a genome or target polynucleotide, means a contiguous sub-region or segment of the genome or target polynucleotide. As used herein, genetic locus, locus, or locus of interest may refer to the position of a nucleotide, a gene or a portion of a gene in a genome or it may refer to any contiguous portion of genomic sequence whether or not it is within, or associated with, a gene, e.g., a coding sequence. A genetic locus, locus, or locus of interest can be from a single nucleotide to a segment of a few hundred or a few thousand nucleotides in length or more. In general, a locus of interest will have a reference sequence associated with it (see description of “reference sequence” below).

The term “reference sequence”, as used herein, refers to a known nucleotide sequence, e.g. a chromosomal region whose sequence is deposited at NCBI's Genbank database or other databases, for example. A reference sequence can be a wild type sequence.

The terms “plurality”, “population” and “collection” are used interchangeably to refer to something that contains at least 2 members. In certain cases, a plurality, population or collection may have at least 10, at least 100, at least 1,000, at least 10,000, at least 100,000, at least 10⁶, at least 10⁷, at least 10⁸ or at least 10⁹ or more members.

The term “variable”, in the context of two or more nucleic acid sequences that are variable, refers to two or more nucleic acids that have different sequences of nucleotides relative to one another. In other words, if the polynucleotides of a population have a variable sequence, then the nucleotide sequence of the polynucleotide molecules of the population may vary from molecule to molecule. The term “variable” is not to be read to require that every molecule in a population has a different sequence to the other molecules in a population.

The term “complexity” refers the total number of different sequences in a population. For example, if a population has 4 different sequences, then that population has a complexity of 4. A population may have a complexity of at least 4, at least 8, at least 16, at least 100, at least 1,000, at least 10,000, at least 100,000, at least 10⁶ (1M), at least 10⁷ (10M), at least 10⁸ (100M) or at least 10⁹ (1B) or more, depending on the desired result.

The term “initial template” refers to a sample that is to be tagmented.

The term “next generation sequencing” refers to the so-called highly parallelized methods of performing nucleic acid sequencing and comprises the sequencing-by-synthesis or sequencing-by-ligation platforms currently employed by Illumina, Life Technologies, Pacific Biosciences and Roche, etc. Next generation sequencing methods may also include, but not be limited to, nanopore sequencing methods such as offered by Oxford Nanopore or electronic detection-based methods such as the Ion Torrent technology commercialized by Life Technologies.

The term “sequence read” refers to the output of a sequencer. A sequence read typically contains a string of Gs, As, Ts and Cs, of 10-1000 or more bases in length and, in many cases, each base of a sequence read may be associated with a score indicating the quality of the base call.

An “oligonucleotide binding site” refers to a site to which an oligonucleotide hybridizes in a target polynucleotide. If an oligonucleotide “provides” a binding site for a primer, then the primer may hybridize to that oligonucleotide or its complement.

The term “strand” as used herein refers to a nucleic acid made up of nucleotides covalently linked together by covalent bonds, e.g., phosphodiester bonds. In a cell, DNA usually exists in a double-stranded form, and as such, has two complementary strands of nucleic acid referred to herein as the “top” and “bottom” strands.

The term “extending”, as used herein, refers to the extension of a primer by the addition of nucleotides using a polymerase. If a primer that is annealed to a nucleic acid is extended, the nucleic acid acts as a template for extension reaction.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide is obtained.

The terms “bridge polymerase chain reaction (PCR)” and “bridge amplification” refer to a solid-phase polymerase chain reaction in which the primers that are extended in the reaction are tethered to a substrate by their 5′ ends. During amplification, the amplicons form a bridge between the tethered primers. Bridge PCR (which may also be referred to as “cluster PCR”) is used in Illumina's Solexa platform. Bridge PCR and Illumina's Solexa platform are generally described in a variety of publications, e.g., Gudmundsson et al (Nat. Genet. 2009 41:1122-6), Out et al (Hum. Mutat. 2009 30:1703-12) and Turner (Nat. Methods 2009 6:315-6), U.S. Pat. No. 7,115,400, and publication application publication nos. US20080160580 and US20080286795. Bridge PCR is done using a lawn of PCRs primers that are tethered to a substrate. A template hybridizes to one of the primers, the primer is extended to produce an extension product, the end of the extension product hybridizes to the other primers, and that primer is extended. The template is amplified and extension products form “bridges”. Bridge PCR is performed on a substrate that has primers that are surface-bound and randomly interspersed with one another (on a molecule-by molecule basis). Such a substrate need not be planer and in certain cases may be in the form of a bead.

The term “clusters” refers to discrete areas of amplification product on a support. In performing bridge PCR, amplification products are produced at sites that are immediately adjacent to the where the original template hybridized to the support. These areas are “clonal” in the sense that each cluster contains a top strand and a bottom strand that is complementary to the top strand and, within each cluster, all of the top strands have the same sequence and all of the bottom strands have the same sequence.

The terms “transposome” and “transposome complex” refer to a complex that is composed of (a) transposase enzyme (which is actually a dimer of transposase polypeptide) and (b) two adapter molecules that each contain a transposon end sequence, where the adapter molecules are bound to the transposase enzyme via the transposon end sequences. In this disclosure, the two adapter molecules that are bound by a transposase may be referred to as the first adapter and the second adapter.

The term “transposase end sequence” refers to a double-stranded sequence to which a transposase (e.g., the Tn5 or Vibhar transposase or variant thereof) binds, where the transposase catalyzes simultaneous fragmentation of a double-stranded DNA sample and tagging of the fragments with sequences that are adjacent to the transposon end sequence (i.e., by “tagmentation”). Transposon end sequences and their use in tagmentation are well known in the art (see, e.g., Picelli et al, Genome Res. 2014 24: 2033-40; Adey et al, Genome Biol. 2010 11:R119 and Caruccio et al, Methods Mol. Biol. 2011 733: 241-55, US20100120098 and US20130203605). The Tn5 transposase recognition sequence is 19 bp in length, although many others are known and are typically 18-20 bp, e.g., 19 bp in length. The transposase recognition sequence of the adaptor may be the transposase recognition sequence of a Tn transposase (e.g. Tn3, Tn5, Tn7, Tn10, Tn552, Tn903), a MuA transposase, a Vibhar transposase (e.g. from Vibrio harveyi), although the transposase recognition sequence for other transposases (Ac-Ds, Ascot-1, Bs1, Cin4, Copia, En/Spm, F element, hobo, Hsmar1, Hsmar2, IN (HIV), IS1, IS2, IS3, IS4, IS5, IS6, IS10, IS21, IS30, IS50, IS51, IS150, IS256, IS407, IS427, IS630, IS903, IS911, IS982, IS1031, ISL2, L1, Mariner, P element, Tam3, Tc1, Tc3, Tel, THE-1, Tn/O, TnA, Tn3, Tn5, Tn7, Tn10, Tn552, Tn903, To11, To12, Tn10, Ty1, including variants thereof) can also be used under certain conditions.

The term “barcode sequence”, as used herein, refers to a unique sequence of nucleotides that can be used to identify and/or track the source of a polynucleotide in a reaction. Barcode sequences may vary widely in size and composition. In particular embodiments, a barcode sequence may have a length in range of from 4 to 60 nucleotides, or from 6 to 50 nucleotides, or from 8 to 40 nucleotides.

The term “randomized” in the context of a template (e.g., an oligonucleotide) that contains a randomized sequence. A randomized sequence may be 4-50 nt in length and may be degenerate in that it may contain at least 4, at least 5, or 6 to 50 or more nucleotides selected from R, Y, S, W, K, M, B, D, H, V, N (as defined by the IUPAC code). In some embodiments, template that has a randomized template may contain a run of 6-50 “Ns”, where N is any nucleotide selected from A, G, T and C.

The terms “symmetrical” and “symmetrically barcoded” refer to a transposome complex in which the pair of adapters have an identical sequence. Specifically, a symmetrically barcoded transposome complex has two copies of the same barcode sequence, one in each of the adapters in the complex.

The terms “asymmetrical” and “asymmetrically barcoded” refer to a transposome complex in which the pair of adapters have different sequences. Specifically, a symmetrically barcoded transposome complex has a pair of adapters that have different barcode sequences.

Other definitions of terms may appear throughout the specification. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a transposome” includes a plurality of transposomes, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Method for Producing a Population of Symmetrically Barcoded Transposome Complexes

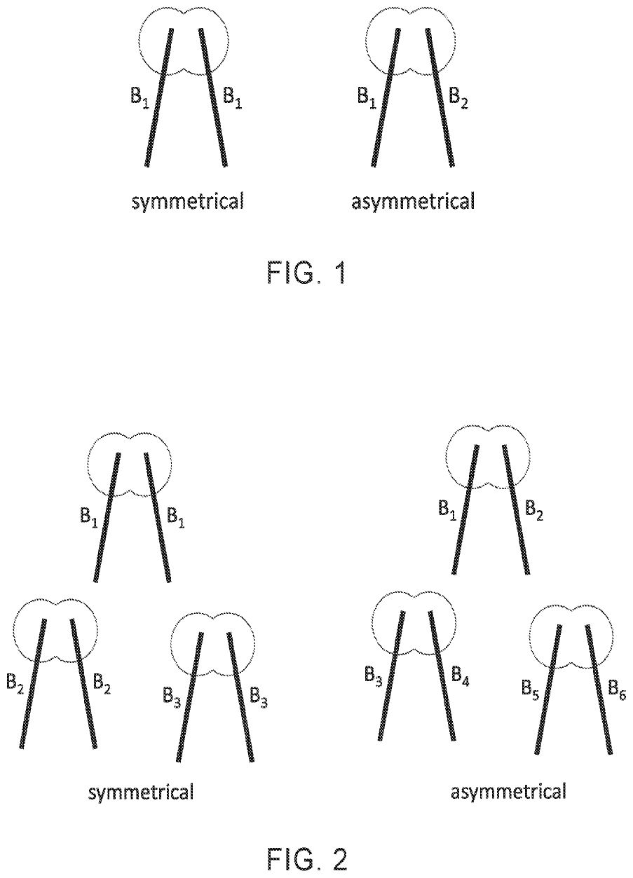

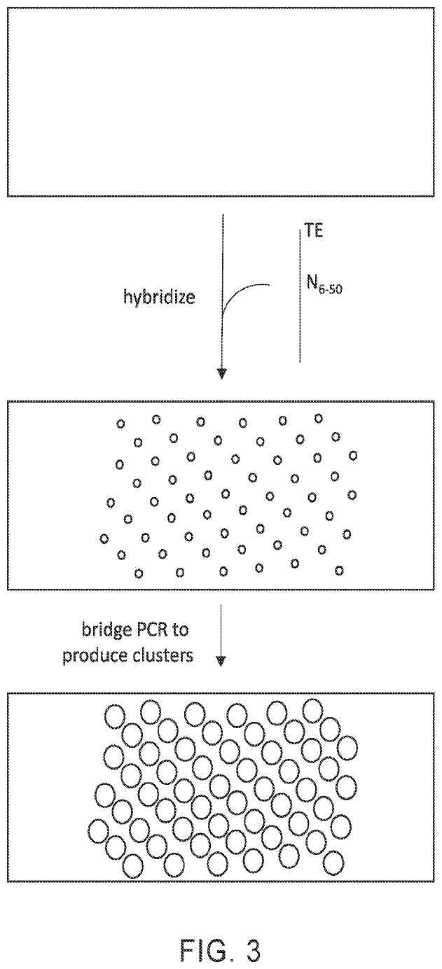

As noted above, provided herein is a method for producing a population of symmetrically barcoded transposome complexes, i.e., a population of transposome complexes in which the barcode varies but the individual transposome complexes are loaded with adapters that have the same barcode sequence. The difference between a symmetrical and an asymmetrical transposome complex is illustrated in FIG. 1. In a symmetrical transposome (shown on the left), the barcode sequence (B₁) is the same in the first and second adapters in the complex. In the asymmetrical transposome shown on the right, the barcode sequences in the adapter are different (i.e., the barcode sequence is B₁ one of the adapter molecules and the barcode sequence is B₂ in the other of the adapter molecules). FIG. 2 illustrates a population of symmetrical transposomes (left) and a population of asymmetrical transposomes. As shown, the individual transposome complexes in the population of symmetrical transposomes (on the left) have symmetrical barcodes (B₁ and B₁, B₂ and B₂, and B₃ and B₃) whereas in the population of asymmetrical transposomes (on the right) have asymmetrical barcodes that vary from adapter to adapter and transposomes to transposome (B₁ and B₂, B₃ and B₄, and B₅ and B₆). The population of symmetrical transposome complexes produced by the present method may have a barcode complexity in the thousands, millions or billions (e.g., at least 1,000, at least 10,000, at least 100,000, at least 1M, at least 10M, at least 100M, or at least 1B), depending on the length of the randomized sequence in the template. Some transposome complexes in the population may have adapters that have the same sequence as other transposome complexes in the population.

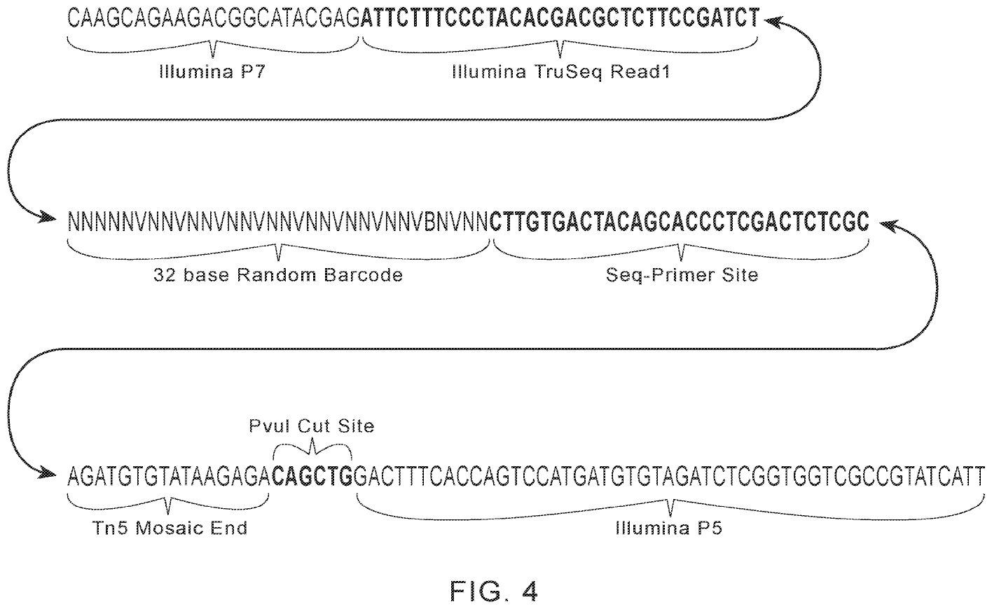

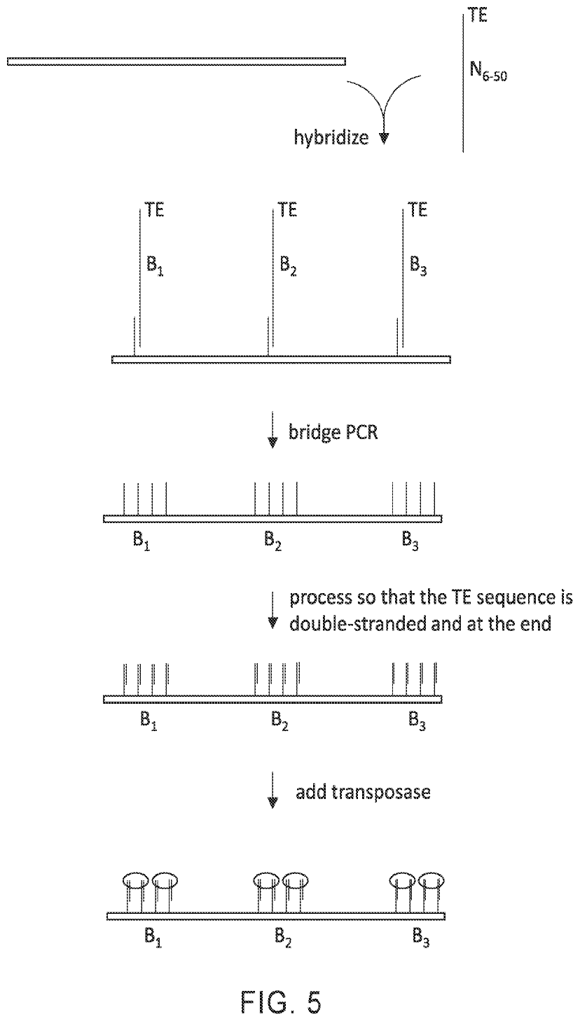

As noted above, this method comprises amplifying a template (i.e., an oligonucleotide) that contains a randomized sequence (which can also be referred to a degenerate sequence) and a transposon end sequence on a solid support by bridge polymerase chain reaction (PCR) to produce uniquely barcoded clusters of single stranded amplification products that are tethered to the support. How clusters are produced in this step of the method is illustrated in FIG. 3. There can be thousands, millions or billions of clusters on the substrate (e.g., at least 1,000, at least 10,000, at least 100,000, at least 1M, at least 10M, at least 100M, or at least 1B clusters), as desired. Depending on the substrate and the amount of oligonucleotide used, the clusters may be in the range of 10,000-2M/mm², e.g., 100,000-1.5M/mm². In some embodiments, substantially all of the clusters (e.g., at least 80%, at least 90%, at least 95% or at least 99.5% of the clusters) may be associated with different barcodes, depending on the complexity of the randomized sequence in the template and other parameters, where randomized sequence in the template becomes the barcode in the individual products. An example of a template oligonucleotide is shown in FIG. 4. As illustrated, the template may contain a sequence (P5) that hybridizes to one of the primers on the substrate, a transposon end sequence (Tn5 ME), a randomized sequence, and a sequence (P7) which is the same as the other of the primers on the substrate, as well as other sequences that could be used in downstream methods. As shown, the template may contain a recognition sequence restriction enzyme that is just 3′ to the transposon end sequence, thereby allowing amplification products to be cleaved at that site to leave the transposon end sequence at the end. The template oligonucleotide does not need to be configured exactly in this way. For example, other sequences can be used instead of P5 and P7, other degenerate regions, and other primer binding sites could be used.

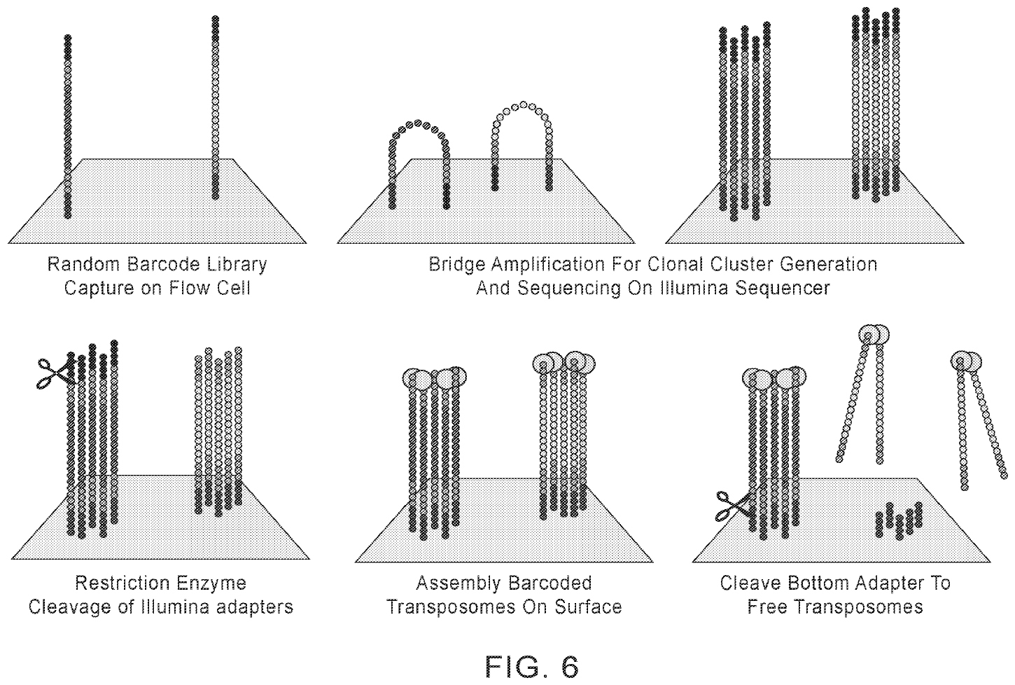

Some of the principles of the next steps of the method are illustrated in FIG. 5. As shown, after bridge PCR, the method involves processing the single-stranded amplification products so that the transposon end sequence is double-stranded and at the end of the products, i.e., so that the amplification products have a proximal end that is tethered to the support, a barcode sequence, and a distal end that comprises a double-stranded transposon end sequence. In some embodiments the entirety of the amplification products may be made double-stranded. In other embodiments, only the transposon end sequence may be made double-stranded.

This step may be done in a variety of different ways. For example, this step may be done by: hybridizing a primer to the single-stranded amplification products, extending the primer using the single-stranded amplification products as a template to produce double stranded products, and then cleaving the ends off the products using a restriction enzyme to leave a double-stranded transposon end sequence at the end. In other embodiments, this step may be done by annealing an oligonucleotide to the single-stranded amplification products (which oligonucleotide hybridizes to the transposon end sequence and then cleaving the ends off the products using a restriction enzyme to leave a double-stranded transposon end sequence at the end. In addition, sequencing the single-stranded amplification products should make the products double-stranded. As such, in some embodiments, the single-stranded amplification products may be sequenced on the support, and a restriction enzyme is used to cleave the end off the products to produce a double-stranded transposon end sequence at the terminus of the cleavage products. In addition to making the amplification products double-stranded, sequencing also allows one to know which barcodes have been made in the method. There are other ways to make the same type of product. For example, if the lawn of primers hybridize to the transposon end sequence in the template then, in theory, the amplification products may contain the transposon end sequence at the end and, as such, they do need to be cleaved. However, in other embodiments (e.g., if an Illumina substrate is used for amplification) the terminal sequence (which corresponds to the sequence of one of the primers sequence on the substrate) may be removed from the amplification products, leaving the transposon end sequence at the end. This may be done by, e.g., making the transposon end sequence as well as at least 4 nt downstream of the transposon end sequence double-stranded (using any of the method described earlier in this paragraph) and cleaving the double-stranded sequence with a restriction enzyme, thereby leaving the transposon end sequence at the end. In these embodiments, the template may be designed to have a recognition site for a restriction enzyme such that when the amplification products are made double-stranded the products can be cleaved with the restriction enzyme to leave a double-stranded transposon end sequence at the end. In addition, a double-stranded oligonucleotide that contains the transposon end sequence could be ligated onto the end of the single stranded amplification product, if desired. In another embodiment, the amplification products may be made double stranded, and then cleaved using a restriction enzyme to leave the transposon end sequence at the end. Next, one strand of the products may removed using an exonuclease and the other strand of the transposon end sequence may be annealed to the products.

The next step of the method involves adding transposase. In these embodiments, the transposase is added to the substrate in a buffer suitable for binding of the transposase to the double-stranded transposon end sequences. As illustrated in FIG. 5, each dimeric unit of transposase binds to two molecules of amplification product that are within the same cluster to produce a symmetrically barcoded transposome. In this example, the method produces six symmetrically barcoded transposomes (two with B₁ and B₁, two with B₂ and B₂, and two with B₃ and B₃). As should be apparent, the complexity of the barcodes in this population may in the thousands, millions or billions, as desired.

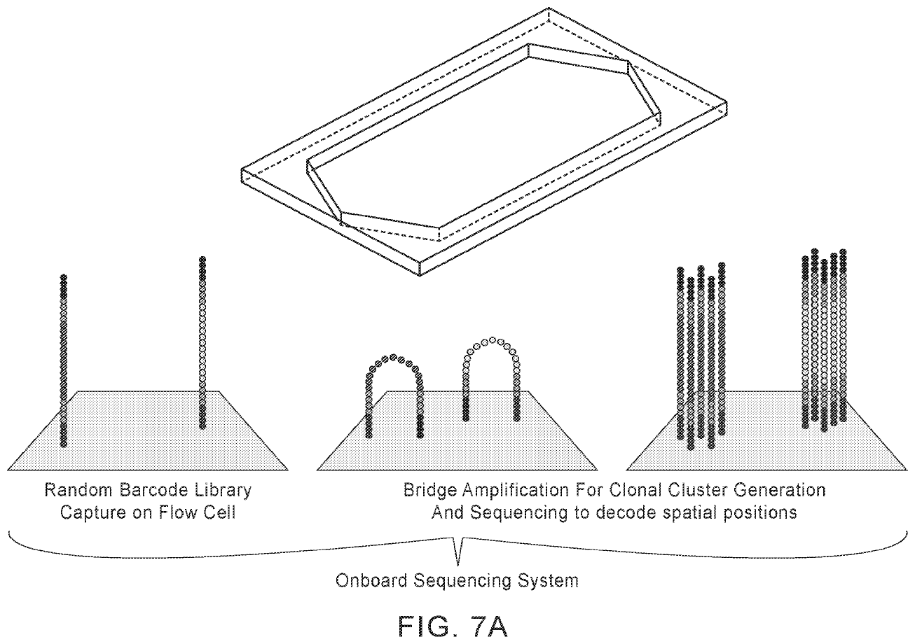

One implementation of the method is illustrated in FIG. 6. In this embodiment, the surface-proximal end of the products can be cleaved using a restriction enzyme to release the transposome complexes from the substrate. This restriction site can be engineered into the template, if desired. Again, if the amplification products are substantially double-stranded the amplification products can be released using a restriction enzyme. Alternatively, the transposome complexes can be released from the substrate by hybridizing an oligonucleotide to the adapters to make the surface proximal part of the adapter double-stranded, and then cleaving the double-stranded region with a restriction enzyme. In theory, clipping of the primer sequences and/or release of the products could be implemented using a nucleic acid-guided endonuclease. Alternatively, the transposome complexes can be released from the substrate by other enzymatic means that are nucleotide, polynucleotide or sequence specific in which the target nucleotide, polynucleotide or sequence is engineered into the template. Alternatively, the nucleotide, polynucleotide or sequence target can be engineered into the solid-phase grafted primer. For example, the primers in the lawn may be synthesized to contain a unique base (e.g., uracil) that can be enzymatically cleaved. These unique bases include uracil (which can be cleaved by UDG or USER enzymes), and 2,6-diamino-4-hydroxy-5-formamidopyrimidine (FapyG) and 8-oxo-7,8-dihydroguanine (8oxoG), which are both cleaved by the enzyme FPG.

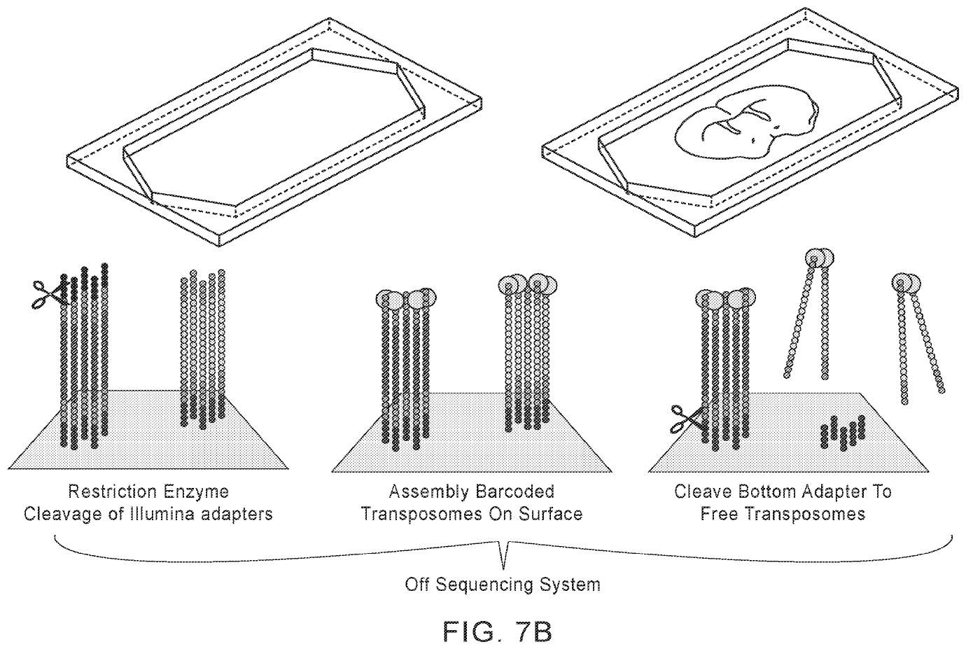

FIG. 7 illustrates how this method can be implemented on a flow cell.

The symmetrical transposomes can be released from the support and used in an in solution tagmentation reaction. Alternatively, the symmetrical transposomes can be left on the support and used to tagment a sample in situ. These embodiments are described in greater detail below.

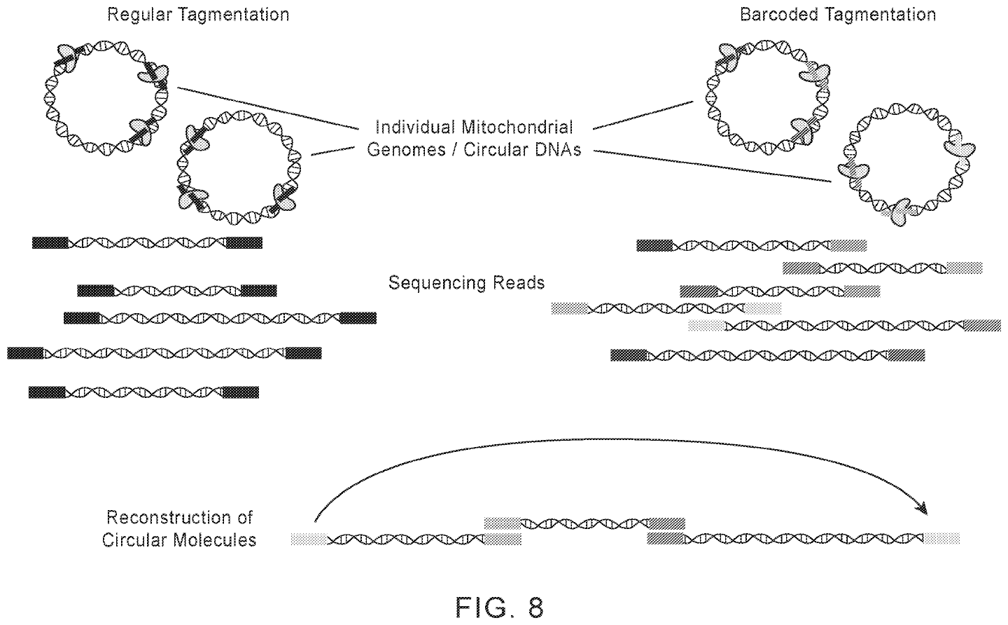

As noted above, in some embodiments the method may further comprise releasing the symmetrically barcoded transposomes from the support. In these embodiments, the transposomes can be collected and used in a tagmentation reaction in a similar way to conventional tagmentation assay (e.g., see, e.g., Caruccio Methods Mol. Biol. 2011 733: 241-55). These tagmentation products can be amplified and sequenced to produce sequence reads corresponding fragments of the sample, appended to a barcode. As illustrated in FIG. 8, because the same barcode is added to adjacent fragments in the tagmentation reaction, the barcodes can be used to assemble the sequences, i.e., to assemble multiple shorter sequences into a longer sequence. There are many ways to assemble sequences. However, in some embodiments, sequence assembly may be done by grouping similar sequence reads by their barcodes, creating a consensus sequence for each group of sequences, then matching pairs of consensus sequences by their barcodes. These sequences should be adjacent to one another in the sample, prior to tagmentation. As such, the method may additionally comprise assembling long fragments (e.g., fragments that are at least 1 kb, at least 10 kb, at least 100 kb, at least 1 MB, at least 10 MB, or entire genomes) from short-read sequences (that are typically under 500 nt in length). However, as illustrated in FIG. 8, this approach is particularly useful for assembling circular molecules (such as the mitochondrial genome or extrachromosomal circular DNA, which is often found in tumor cells).

As noted above, in some embodiments, the method may involve sequencing the unique barcodes of each of the clusters on the support prior to adding the transposase. In these embodiments, the sequences may be compiled into a table and used to confirm barcodes that have been identified in downstream steps. For example, after tagmentation and sequencing, any barcode identified in the sequence reads from the sample can be compared to the barcode table to confirm that it is, indeed, one of the expected barcodes.

In some embodiments, the sequencing reaction may provide at least two pieces of data: the sequence of the barcode in each cluster and a spatial coordinate for each clusters (e.g., in x, y coordinates). In these embodiments, a barcode sequence may be associated with the coordinates on the substrate. In these embodiments, the method may further comprise placing a planar biological sample, e.g., a tissue section, on the support and performing a tagmentation reaction on the sample while it is on the support. In these embodiments, the biological sample may be placed on the support and nucleic acids from the sample (e.g., cDNA/RNA hybrids, cDNA/cDNA hybrids or genomic DNA, etc.) may move from the sample to the support by diffusion or electrophoresis. These molecules will become attached to the substrate after they are tagmented. In some embodiments, the biological sample may be placed on the support and the transposomes may be released from the support and then travel into the sample. In some embodiments, the in-situ tagmented nucleic acid is nuclear chromosomal DNA. In other embodiments the in-situ tagmented nucleic acid nuclear extrachromosomal DNA. In other embodiments the in-situ tagmented nucleic acid is extra-nuclear, including, but not limited to mitochondrial DNA, plastid DNA or pathogenic DNA. In some embodiments, the in-situ tagmented nucleic acid is in the form of chromatin, in which tagmentation insertion sites delimit regions of accessible chromatin.

In cases where the tagmentation products are attached to the support, the products (with the barcode) may be amplified on the support, and the amplification products may be collected and then sequenced (using another substrate, if they are sequenced by the Illumina method). In these embodiments, the sequences can be mapped to sites on the support by their barcodes. As such, these embodiments of the method may comprise mapping a sequence to a site on the support using the barcode associated with the sequence as well as the spatial coordinates for that barcode. This method can be used to construct an image of the sample, where the image corresponds to sequences obtained in the sequencing reaction.

The in situ tagmentation methods above-described method can be used to analyze cells from a subject to determine, for example, whether the cell is normal or not, to determine whether the cells are responding to a treatment, or to examination cell lineage. For example, if the tissue section is a section tumor, at least some of the data collected should be from a tumor cell, and this data can be mapped to a particular site in the tissue section. In these embodiments, the method may be employed to determine the degree of dysplasia in cancer cells. A biological sample may be isolated from an individual, e.g., from a soft tissue or from a bodily fluid, or from a cell culture that is grown in vitro. A biological sample may be made from a soft tissue such as brain, adrenal gland, skin, lung, spleen, kidney, liver, spleen, lymph node, bone marrow, bladder stomach, small intestine, large intestine or muscle, etc. Bodily fluids include blood, plasma, saliva, mucous, phlegm, cerebral spinal fluid, pleural fluid, tears, lactal duct fluid, lymph, sputum, cerebrospinal fluid, synovial fluid, urine, amniotic fluid, and semen, etc. Biological samples also include cells grown in culture in vitro. A cell may be a cell of a tissue biopsy, scrape or lavage or cells. In particular embodiments, the cell may in a formalin fixed paraffin embedded (FFPE) sample. In particular cases, the method may be used to distinguish different types of cancer cells in FFPE samples.

In addition, the method may further comprise staining and imaging the sample prior to its removal from the substrate. For example, in addition to the labeling methods described above, the sample may be stained using a cytological stain, either before or after performing the method described above. In these embodiments, the stain may be, for example, phalloidin, gadodiamide, acridine orange, bismarck brown, barmine, Coomassie blue, bresyl violet, brystal violet, DAPI, hematoxylin, eosin, ethidium bromide, acid fuchsine, haematoxylin, hoechst stains, iodine, malachite green, methyl green, methylene blue, neutral red, Nile blue, Nile red, osmium tetroxide (formal name: osmium tetraoxide), rhodamine, safranin, phosphotungstic acid, osmium tetroxide, ruthenium tetroxide, ammonium molybdate, cadmium iodide, carbohydrazide, ferric chloride, hexamine, indium trichloride, lanthanum nitrate, lead acetate, lead citrate, lead(II) nitrate, periodic acid, phosphomolybdic acid, potassium ferricyanide, potassium ferrocyanide, ruthenium red, silver nitrate, silver proteinate, sodium chloroaurate, thallium nitrate, thiosemicarbazide, uranyl acetate, uranyl nitrate, vanadyl sulfate, or any derivative thereof. The stain may be specific for any feature of interest, such as a protein or class of proteins, phospholipids, DNA (e.g., dsDNA, ssDNA), RNA, an organelle (e.g., cell membrane, mitochondria, endoplasmic recticulum, golgi body, nuclear envelope, and so forth), or a compartment of the cell (e.g., cytosol, nuclear fraction, and so forth). The stain may enhance contrast or imaging of intracellular or extracellular structures. In some embodiments, the sample may be stained with DAPI or hematoxylin and eosin (H&E). In such methods, sequencing data may be superimposed onto the image of the tissue section in order to observe correlations between a cells' genotype and phenotype.

Also provided is a substrate comprising clusters of transposomes, wherein each transposome comprises: (a) two identical molecules of amplification product that each have a proximal end that is tethered to the support, a barcode sequence, and a distal end that comprises a double-stranded transposon end sequence, and (b) a transposase, wherein the transposase is bound to the transposon end sequences of the two molecules of amplification product. In these embodiments, the barcode sequence is the same for all of the transposomes within a cluster but different between clusters. There can be thousands, millions or billions of clusters on the substrate (e.g., at least 1,000, at least 10,000, at least 100,000, at least 1M, at least 10M, at least 100M, or at least 1B clusters), each with a different barcode.

Also provided is a population of symmetrically barcoded transposomes, wherein each transposome comprises a transposase and two identical molecules of nucleic acid that each comprise a barcode sequence and a double-stranded transposon end sequence. The population may comprise at least 1,000 different barcodes (e.g., at least 1,000, at least 10,000, at least 100,000, at least 1M, at least 10M, at least 100M, or at least 1B barcodes). Kits comprising this population of transposomes composition are also provided.

The substrate and population of transposomes may be used for tagmenting a nucleic acid sample, which methods may comprise combining the nucleic acid sample with the population of transposomes and a divalent cation to produce a reaction mix and incubating the reaction mix to tagment the nucleic acid sample. As would be apparent, the resulting tagmentation products can be sequenced to produce sequences of fragments that are appended to a barcode. A described above, the fragments may be assembled into a longer sequence (which, in some embodiments, may be a circular molecule) using the barcodes. In some embodiments, the method may comprise producing an image of the sample using the sequencing data.

This method may be employed to analyze genomic DNA, cDNA/RNA hybrids, and double-stranded cDNA from virtually any organism, including, but not limited to, plants, animals (e.g., reptiles, mammals, insects, worms, fish, etc.), tissue samples, bacteria, fungi (e.g., yeast), phage, viruses, cadaveric tissue, archaeological/ancient samples, etc. In certain embodiments, the genomic DNA used in the method may be derived from a mammal, wherein in certain embodiments the mammal is a human. In exemplary embodiments, the sample may contain genomic DNA from a mammalian cell, such as, a human, mouse, rat, or monkey cell. The sample may be made from cultured cells or cells of a clinical sample, e.g., a tissue biopsy, scrape or lavage or cells of a forensic sample (i.e., cells of a sample collected at a crime scene). In particular embodiments, the nucleic acid sample may be obtained from a biological sample such as cells, tissues, bodily fluids, and stool. Bodily fluids of interest include but are not limited to, blood, serum, plasma, saliva, mucous, phlegm, cerebral spinal fluid, pleural fluid, tears, lactal duct fluid, lymph, sputum, synovial fluid, urine, amniotic fluid, and semen. In particular embodiments, a sample may be obtained from a subject, e.g., a human.

In some embodiments, the sample comprises DNA fragments obtained from a clinical sample, e.g., a patient that has or is suspected of having a disease or condition such as a cancer, inflammatory disease or pregnancy. In some embodiments, the sample may be made by extracting fragmented DNA from an archived patient sample, e.g., a formalin-fixed paraffin embedded tissue sample. In other embodiments, the patient sample may be a sample of cell-free circulating DNA from a bodily fluid, e.g., peripheral blood. The DNA fragments used in the initial steps of the method should be non-amplified DNA that has not been denatured beforehand. In other embodiments, the DNA in the sample may already be partially fragmented (e.g., as is the case for FFPE samples and circulating cell-free DNA (cfDNA), e.g., ctDNA).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

Generation of Barcoded Transposomes within Glass Flow Cell

100 pM random-barcode Tn5 template (CAAGCAGAAGACGGCATACGAGATTCTTTCCCTACACGACGCTCTTCCGATCTNNN NNVNNVNNVNNVNNVNNVNNVNNVBNVNNCTTGTGACTACAGCACCCTCGACTCT CGCAGATGTGTATAAGAGACAGCTGGACTTTCACCAGTCCATGATGTGTAGATCTC GGTGGTCGCCGTATCATT (SEQ ID NO: 1; FIG. 4) was loaded into an Illumina MiSeq, using a MiSeq Nano Kit v2, following the manufactures instructions. Following on-system cluster generation, single read sequencing was run using a custom sequencing primer complementary to the barcode 3′-adjacent sequence in the random barcode library to read out the barcode sequence of each cluster. Following sequencing, the flow cell was removed, and the flow cell channel was flushed three times with 8 μl water, before loading with 101 Pvul-HF restriction enzyme cocktail (8.5 μl water, 1 μl 10× CUTSmart buffer (NEB), 0.5 μl Pvul-HF (NEB)) and sealing the flow-cell ports with Microseal B PCR Plate Sealing Film (BIORAD). The sealed flow cell was then incubated at 37° C. overnight on a flat block thermocycler. Following incubation, the flow cell channel was flushed once with water before loading 101 exonuclease I mix (8.5 μl water, 1 μl 10× exonuclease I buffer (NEB), 0.5 μl exonuclease I (NEB)) and incubated at 37° C. for 45 minutes. The flow cell channel was then washed three times with 0.1N NaOH to denature double stranded DNA sequencing products, before neutralizing with three washes of 0.1M Tris-HCl pH 7.5. 10 μl of 10 μM Tn5 Universal Mosaic End sequence was then flowed through the flow-cell channel and hybridized to the Tn5 adapter clusters by incubating at 4° C. for 30 minutes. The hybridized DNA was washed 3 times with cold water before flowing 10 μl 6 μM purified Tn5 transposase through the flow cell channel. Transposome complexes were allowed to form by incubating the flow cell at 23° C. for 30 minutes. Unbound Tn5 was then washed away with three washes with cold water.

### Example 2

Tagmentation of DNA with Surface Linked Barcoded Tn5 within Glass Flow Cell

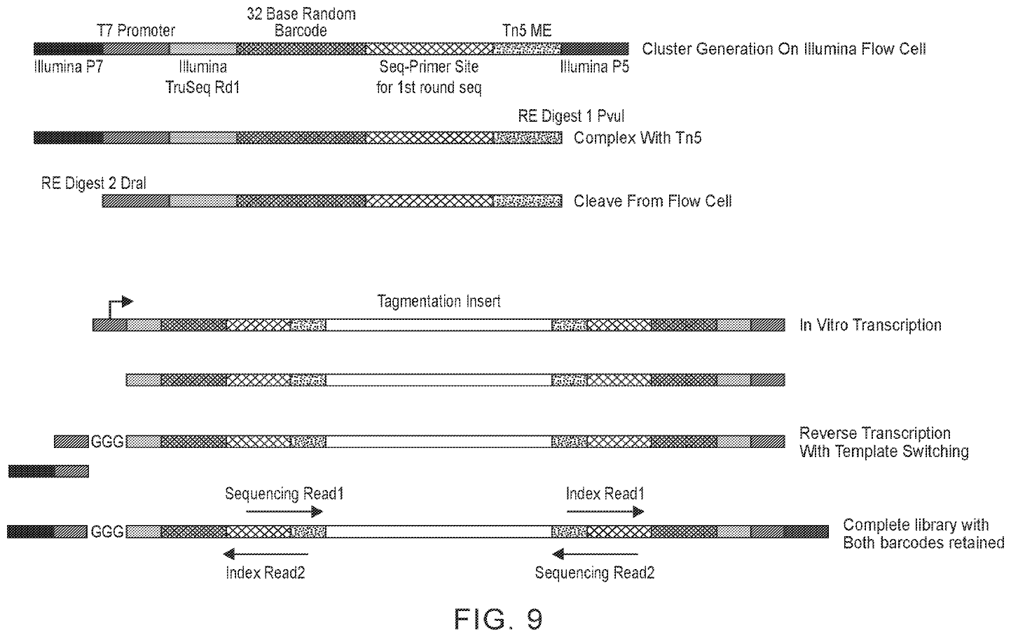

Following transposome assembly on the flow cell surface, 10 ng of purified genomic DNA in 1× tagmentation buffer (10 mM TAPS-NaOH at pH 8.5 [RT], 5 mM MgCl2, 10% DMF) with 2 μM Illumina Nextera-P7-adapter loaded Tn5 was flowed into the flow-cell channel and tagmented by incubation at 37° C. for 1 hour on a flat block thermocycler. Following tagmentation, bound transposase wash washed off the DNA fragments by washing three times with 0.1% SDS, followed by three times with cold water. Tagmented, surface bound DNA was then amplified using NEBNext High-Fidelity 2×PCR Master Mix (NEB) on a flat block thermocycler using primers to the surface bound Illumina TruSeq P5 sequence and Illumina Nextera P7 sequence. PCR product wash collected through the flow-cell channel port and quantified by Bioanalyzer High-Sensitivity DNA Analysis assay (Agilent) before sequencing on an Illumina sequencer. FIG. 9 shows the structure of various starting products, intermediate reaction products, and products, that may be used or made in practicing this method.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for producing a population of symmetrically barcoded transposomes, comprising:
(a) amplifying a template that contains a randomized sequence and a transposon end sequence on a solid support by bridge polymerase chain reaction (PCR) to produce uniquely barcoded clusters of single-stranded amplification products that are tethered to the support;
(b) processing the single-stranded amplification products so that the transposon end sequence is double-stranded and at the end of the products; and
(c) adding transposase to the support under conditions by which the transposase binds to the double-stranded transposon end sequences, to produce the population of symmetrically barcoded transposomes.

2. The method of claim 1, wherein step (b) is done by extending a primer using the single stranded amplification products as a template to produce double stranded products and then cleaving the ends off the products using a restriction enzyme, or by annealing an oligonucleotide to the single-stranded amplification products and then cleaving the ends off the products using a restriction enzyme, or by sequencing the stranded amplification products and then cleaving the ends off the products using a restriction enzyme.

3. The method of claim 1, further comprising releasing the population of symmetrically barcoded transposomes from the support after step (c).

4. The method of claim 3, further comprising performing a tagmentation reaction using the released transposomes.

5. The method of claim 4, further comprising sequencing the tagmentation products to produce sequence reads, wherein the sequence reads comprise sequences of fragments that comprise a barcode derived from the randomized sequence.

6. The method of claim 5, further comprising assembling the sequences of multiple fragments into a longer sequence using the barcodes.

7. The method of claim 1, further comprising sequencing the unique barcodes of each of the clusters between steps (a) and (c).

8. The method of claim 7, wherein the sequencing provides a spatial coordinate for each the sequenced barcodes, and the method further comprises performing a tagmentation reaction on a planar biological sample that is placed on the support, with releasing the barcoded transposomes from the support, and then sequencing the tagmentation products, or an amplification product thereof.

9. The method of claim 8, wherein tagmentation products are tethered to the support, and the method comprises amplifying the tagmentation products, collecting the amplification products, and sequencing the amplification products.

10. The method of claim 9, further comprising mapping a sequence to a site on the support using the barcode associated with the sequence and the spatial coordinates for that barcode.

11. The method of claim 10, further comprising constructing an image of the sample.

12. A substrate comprising clusters of transposomes, wherein each transposome comprises:
(a) two identical molecules of amplification product that each have a proximal end that is tethered to the support, a barcode sequence, and a distal end that comprises a double-stranded transposon end sequence,
wherein the barcode sequence is the same for the transposomes within a cluster but different for transposomes in different clusters; and
(b) a transposase, wherein the transposase is bound to the transposon end sequences of the two molecules of amplification product of (a).

13. The substrate of claim 12, wherein the substrate comprises at least 1M of said clusters.

14. A population of symmetrically barcoded transposomes, wherein each transposome comprises:
a transposase and
two identical molecules of nucleic acid that each comprise a barcode sequence and a double-stranded transposon end sequence,
wherein the population comprises at least 1,000 different barcode sequences.

15. The population of claim 14, wherein the population comprises at least 1M different barcode sequences.

16. A method comprising tagmenting a nucleic acid sample, comprising:
combining the nucleic acid sample with the substrate or population of claim 12 and a divalent cation to produce a reaction mix; and
incubating the reaction mix to tagment the nucleic acid sample.

17. The method of claim 16, further comprising sequencing the tagmentation products to produce sequences of fragments appended to a barcode.

18. The method of claim 17, further comprising assembling multiple fragment sequences into a longer sequence using the barcodes.

19. The method of claim 18, wherein the assembled sequence is a circular molecule.


SEQUENCE LISTING (1 sequence)

SEQ ID NO: 1 - DNA, 185 bp, Unknown
  100pM random-barcode transposon end sequence (Tn5) template
  misc_feature (54)..(58): n is a, c, g, or t
  misc_feature (60)..(61): n is a, c, g, or t
  misc_feature (63)..(64): n is a, c, g, or t
  misc_feature (66)..(67): n is a, c, g, or t
  misc_feature (69)..(70): n is a, c, g, or t
  misc_feature (72)..(73): n is a, c, g, or t
  misc_feature (75)..(76): n is a, c, g, or t
  misc_feature (78)..(79): n is a, c, g, or t
  misc_feature (82)..(82): n is a, c, g, or t
  misc_feature (84)..(85): n is a, c, g, or t
caagcagaagacggcatacgagattctttccctacacgacgctcttccgatctnnnnnvnnvnnvnnvnnvnnvnnvnnvbnvnncttgtgactacagcaccctcgactctcgcagatgtgtataagagacagctggactttcaccagtccatgatgtgtagatctcggtggtcgccgtatcatt